(19) United States
(12) Patent Application Publication
(10) Pub. No.: US 2024/0238191 A1
(43) Pub. Date: Jul. 18, 2024

(54) PERSONAL CARE COMPOSITION

(71) Applicant: The Procter & Gamble Company, Cincinnati, OH (US)

(72) Inventors: Howard David Hutton, III, Oregonia, OH (US); Karla Jean Siereveld, Liberty Township, OH (US)

(21) Appl. No.: 18/609,690

(22) Filed: Mar. 19, 2024

Related U.S. Application Data
(63) Continuation-in-part of application No. 17/147,574, filed on Jan. 13, 2021, now abandoned.
(60) Provisional application No. 62/960,281, filed on Jan. 13, 2020.

(51) Int. Cl. A61K 8/9794; A61K 8/04; A61K 8/34; A61K 8/368; A61K 8/44; A61K 8/46; A61K 8/73; A61K 8/892; A61Q 5/02; A61Q 5/12

(52) U.S. Cl. CPC A61K 8/9794; A61K 8/042; A61K 8/342; A61K 8/368; A61K 8/44; A61K 8/442; A61K 8/466; A61K 8/731; A61K 8/892; A61Q 5/02; A61Q 5/12; A61K 2800/30

(57) ABSTRACT

A method of making a sulfate-free, multi-phase personal care composition that exhibits excellent cleaning qualities. The method includes combining a fatty alcohol, a sulfate-free gel network surfactant and water, with our without heating, such that the gel network surfactant and the water partition into the fatty alcohol to form a pre-mix, which is cooled below the chain melt temperature of the fatty alcohol to form a gel network. The gel network is added to a liquid carrier alone with a sulfate-free detersive surfactant and aloe vera. The aloe vera contains 100 mg/100 g to 300 mg/100 g of salt. The ingredients are then mixed to form the multi-phase personal care composition that provides improved hair strength and an anti-oxidant benefit.

### FIELD

The present disclosure generally relates to a method of making a multi-phase personal care compositions free of sulfated surfactants and method of using the same. More specifically, the present disclosure relates to a method of making a multi-phase personal care composition that cleans hair and delivers potentiated aloe vera for increased hair conditioning benefits.

### BACKGROUND

Human hair becomes soiled due to contact with the surrounding environment and from sebum secreted by the scalp. Soiled hair has a dirty feel and exhibits an unattractive appearance. Application and washing of the soiled hair with a shampoo composition can restore hair to a clean and attractive appearance by removing oil and other soils from the hair. Known shampoo compositions typically remove oil and soil from hair through inclusion of sulfated surfactants. Shampoos including sulfated surfactants, such as sodium lauryl sulfate and sodium laureth sulfate, however, exhibit a number of undesirable characteristics such as poor-quality hair feel and hair and skin dryness after washing due to the harshness of the sulfated surfactants. Shampoos including sulfated surfactants also face poor consumer acceptance as a consequence of this harshness.

In addition to cleaning, some users also desire a shampoo that can deliver a hair health or appearance benefit other than cleaning. For example, some users may desire a shampoo that includes a benefit agent for making hair appear soft, shiny, and manageable. And some consumers may prefer a hair care composition that includes a benefit agent for nourishing the hair, reducing frizz, and/or improving hair texture.

Consequently, it would be desirable to provide personal care compositions which can provide acceptable cleaning quality to soiled hair, while additionally delivering consumer desired hair health or appearance benefits.

### SUMMARY

The present disclosure is directed to a method of making a multi-phase personal care composition. The method comprises combining a fatty alcohol, a sulfate-free gel network surfactant and water such that the gel network surfactant and the water partition into the fatty alcohol to form a pre-mix; cooling the pre-mix below the chain melt temperature of the fatty alcohol to form a gel network; adding the gel network to a liquid carrier comprising water; adding a sulfate-free detersive surfactant to the liquid carrier; adding about 0.01% to about 0.05% aloe vera to the liquid carrier, based on the weight of the personal care composition, wherein the aloe vera comprises about 100 mg/100 g to about 300 mg/100 g of a salt selected from sodium salt, potassium salt, calcium salt, zinc salt, magnesium salt, copper salt and mixtures thereof; adding about 0.1 wt % to about 3 wt % of a cationic cellulose polymer, based on the weight of the personal care composition; and mixing the liquid carrier, gel network, detersive surfactant, aloe vera and cationic cellulose polymer to form the multi-phase personal care composition.

### DETAILED DESCRIPTION

All percentages are by weight of the total composition and all ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at 25° C. and at ambient conditions, where “ambient conditions” means one atmosphere of pressure and 50% relative humidity. All such weights as they pertain to listed ingredients are based on the active level and do not include carriers or by-products that may be included in commercially available materials, unless otherwise specified.

“Charge density” means the ratio of the number of positive charges on a polymer to the molecular weight of said polymer.

“Comprising,” as used herein, means that other steps and other ingredients which do not affect the end result can be added. This term encompasses the terms “consisting of” and “consisting essentially of.” The compositions and methods/processes of the present disclosure can comprise, consist of, and consist essentially of the elements and limitations of the invention described herein, as well as any of the additional or optional ingredients, components, steps, or limitations described herein.

The term “polymer,” as used herein, includes materials whether made by polymerization of one type of monomer or made by two (i.e., copolymers) or more types of monomers.

The term “suitable for application to human hair,” as used herein, means that the personal care compositions or components thereof, are acceptable for use in contact with human hair and the scalp and skin without undue toxicity, incompatibility, instability, allergic response, and the like.

The term “water soluble,” as used herein, means that the material is soluble in water. In certain embodiments, the material can be soluble at 25° C. at a concentration of 0.1% by weight of the water solvent, in certain embodiments at 1% by weight of the water solvent, in certain embodiments at 5% by weight of the water solvent, and in certain embodiments at 15% or more by weight of the water solvent.

The terms “sulfate-free” and “substantially free of sulfates” means essentially free of sulfate-containing compounds except as otherwise incidentally incorporated as minor components.

The term “sulfated surfactants” means surfactants which contain a sulfate group. The term “substantially free of sulfated surfactants” means essentially free of surfactants containing a sulfate group except as otherwise incidentally incorporated as minor components.

### Personal Care Compositions

As will be described herein, a personal care composition is disclosed which exhibits excellent cleaning qualities without the use, or inclusion, of a sulfated surfactant. Generally, the personal care compositions described herein contain from about 0.01 wt % to about 0.05 wt % of aloe, from about 0.02 wt % to about 0.05 wt % of aloe, from about 0.02 wt % to about 0.04 wt % of aloe, from about 0.03 wt % to about 0.04 wt % of aloe. The aloe vera can contain salts, which can further increase aloe vera potentiation, making the aloe vera more available to deliver consumer desired benefits such as increased hair strength as demonstrated by a decrease in hair breakage during washing. The aloe vera can contain from about 100 mg/100 g (of aloe) to about 300 mg/100 g (of aloe) of a material selected from the group consisting of sodium salt, potassium salt, calcium salt, zinc salt, magnesium salt, copper salt and mixtures thereof. Additionally, the personal care compositions described herein contain a cationic cellulose polymer. The polymer can be included by weight of the personal care composition at about 0.1 wt % to about 3 wt %, about 0.1 wt % to about 2.0 wt %, and at about 0.1 wt % to about 1.0 wt %. The cationic cellulose polymer can have a weight average molecular weight of about 1 million g/mol to about 2.2 million g/mol, (e.g., about 1.8 million g/mol to about 2 million g/mol); and a charge density of from about 0.4 to about 2.6, from about 0.4 to about 2, and from about 0.6 to about 1.6. Suitable polymers include JR 30 M and LR 400 available from Amerchol Corporation.

According to certain embodiments, the personal care compositions described herein can be free of sulfated surfactants and can include a detersive surfactant, a cationic cellulose, aloe, and a liquid carrier.

### Detersive Surfactant

According to certain embodiments, the personal care compositions described herein can include one or more detersive surfactants. As can be appreciated, surfactants provide a cleaning benefit to soiled hair and hair follicles by facilitating the removal of oil and other soil components from the soiled hair. Surfactants generally facilitate such cleaning due to their amphiphilic nature which allows for the surfactants to break up, and form micelles around, oil and other soils in the hair which can then be rinsed out, thereby removing them from the hair.

Traditional shampoo compositions generally include sulfated surfactants such as sodium lauryl sulfate, sodium laureth sulfate, ammonium lauryl sulfate, and ammonium laureth sulfate to provide a cleaning benefit. Although sulfated surfactants are effective at removing oil and other soils from hair and skin surfaces of a consumer, such sulfated surfactants also remove beneficial oils. Hairs and skins washed with such sulfated surfactants can be unattractive and can have a dry feel to consumers. The personal care compositions described herein can be substantially free of any sulfated surfactants and can alternatively include milder detersive surfactants. As used herein, “detersive surfactant” means a surfactant substantially free of sulfates.

According to certain embodiments, suitable detersive surfactants can instead be selected from one or more anionic surfactants, cationic surfactants, amphoteric surfactants, zwitterionic surfactants, and non-ionic surfactants which are substantially free of sulfates. Such surfactants should generally be physically and chemically compatible with the other components of the personal care compositions described herein and should not otherwise unduly impair product stability, aesthetics, or performance.

### A. Anionic Surfactants

Examples of suitable anionic detersive surfactants for use in the personal care compositions described herein can include those which are known for use in hair care or other personal care compositions including, for example, isethionate, sarcosinate, sulfonate, sulfosuccinate, sulfoacetate, glycinate, glutamate, glucosecarboxylate, and phosphate ester surfactants.

In certain embodiments, suitable anionic surfactants can include water-soluble olefin sulfonates which have the general formula R¹—SO₃M where R¹ is a straight or branched chain, saturated, aliphatic hydrocarbon radical having from 10 to 24 carbon atoms, 10 to 18 carbon atoms, or from 13 to 15 carbon atoms; and M is a water soluble cation such as ammonium, sodium, potassium, triethanolamine cation, or salts of the divalent magnesium ion with two anionic surfactant anions. Suitable olefin sulfonates such as sodium paraffin sulfonates can be produced through the reaction of SO₂ and O₂ with a suitable chain length paraffin.

In certain embodiments, suitable olefin sulfonates can also contain minor amounts of other materials, such as alkene disulfonates depending upon the reaction conditions, proportion of reactants, the nature of the starting olefins and impurities in the olefin stock and side reactions during the sulfonation process. Examples of additional olefin sulfonates and mixtures thereof are described in U.S. Pat. No. 3,332,880, which is incorporated herein by reference.

Another class of suitable anionic detersive surfactants includes the beta-alkyloxy alkane sulfonates. Beta-alkyloxy alkane sulfonates surfactants conform to Formula I:

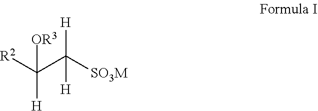

where R² is a straight chain alkyl group having from about 6 to about 20 carbon atoms, R³ is a lower alkyl group having from about 1 to about 3 carbon atoms, preferably 1 carbon atom, and M is a water-soluble cation as previously described in the water-soluble olefin sulfonates.

In certain embodiments, suitable anionic detersive surfactants can include isethionate surfactants. For example, suitable isethionate surfactants can include the reaction product of fatty acids esterified with isethionic acid and neutralized with sodium hydroxide. In certain embodiments, suitable fatty acids for isethionate surfactants can be derived from coconut oil or palm kernel oil including amides of methyl tauride. Additional examples of suitable isethionic anionic surfactants are described in U.S. Pat. Nos. 2,486,921; 2,486,922; and 2,396,278, each of which is incorporated herein by reference.

In certain embodiments, detersive anionic surfactants can be succinate surfactants. Examples of suitable succinate surfactants can include disodium N-octadecylsulfo succinnate, disodium lauryl sulfosuccinate, diammonium lauryl sulfosuccinate, laureth sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, and dioctyl esters of sodium sulfosuccinic acid.

In certain embodiments, suitable anionic detersive surfactants can include one or more of sodium cocoyl isethionate (“SCI”), sodium lauroyl methyl isethionate (“SLMI”), sodium lauroyl sarcosinate, sodium C₁₂-C₁₄ olefin sulfonate, sodium lauroyl glycinate, sodium cocoamphoacetate, sodium cocoyl glutamate, sodium lauryl glucosecarboxylate, sodium lauryl sulfosuccinate, sodium laureth sulfosuccinate, sodium lauryl sulfoacetate, lauryl sarcosine, cocoyl sarcosine, sodium methyl lauroyl taurate, sodium methyl lauroyl taurate, sodium tridecyl benzene sulfonate, sodium dodecyl benzene sulfonate, phosphate ester surfactants, and fatty acid surfactants.

### B. Cationic Surfactants

In certain embodiments, a suitable detersive surfactant can be a cationic surfactant described by Formula II:

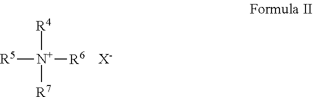

wherein at least one of R⁴, R⁵, R⁶ and R⁷ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, and wherein the remainder of R⁴, R⁵, R⁶ and R⁷ are independently selected from an aliphatic group having from 1 to 22 carbon atoms or an aromatic, alkoxy, polyoxyalkylene, alkylamido, hydroxyalkyl, aryl or alkylaryl group having up to 22 carbon atoms; and X⁻ is a salt-forming anion such as a halogen (e.g. chloride, bromide), acetate, citrate, lactate, glycolate, phosphate, nitrate, sulfonate, alkylsulfate, and alkyl sulfonate radicals. The aliphatic groups can contain, in addition to carbon and hydrogen atoms, ether linkages, and other groups such as amino groups. The longer chain aliphatic groups, e.g., those of 12 carbon atoms, or higher, can be saturated or unsaturated. In certain embodiments, R⁴, R⁵, R⁶ and R⁷ are independently selected from C₁ to C₂₂ branched or straight alkyl or alkenyl groups. Specific examples of cationic surfactants can include compounds having the following Cosmetic, Toiletries, and Fragrance Association (“CTFA”) designations: quaternium-8, quaternium-14, quaternium-18, quaternium-18 methosulfate, quaternium-24, and mixtures thereof.

In certain embodiments, suitable cationic surfactants of Formula II can include at least one alkyl chain having at least 16 carbon atoms. Examples of such surfactants can include: behenyl trimethyl ammonium chloride available, for example, with tradename INCROQUAT TMC-80 from Croda and ECONOL TM22 from Sanyo Kasei; cetyl trimethyl ammonium chloride available, for example, with tradename CA-2350 from Nikko Chemicals, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride.

In certain embodiments, a primary, secondary, or tertiary fatty amine cationic surfactant can be selected. Particularly useful are tertiary amido amines having an alkyl group including 12 to 22 carbon atoms. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with about 5 moles of ethylene oxide) stearylamine, dihydroxyethylstearylamine, isostearylamidopropyldimethylamine, oleamidopropyldimethylamine, cocamidopropyldimethylamine and arachidylbehenylamine.

In certain embodiments, suitable cationic amine surfactants can include bis-hydroxyethyl lauryl amine, lauryl dimethylamine, lauroyl dimethyl amidoproplyl amine, cocoylamidopropyl amine, and the like. Additional amine surfactants are disclosed in U.S. Pat. No. 4,275,055 which is incorporated by reference herein.

In certain embodiments, amines can be used in combination with acids such as l-glutamic acid, lactic acid, hydrochloric acid, malic acid, succinic acid, acetic acid, fumaric acid, tartaric acid, citric acid, maleic acid, and mixtures thereof. In such embodiments, the amine can be partially neutralized with such acids at a molar ratio of the amine to the acid of from about 1:0.3 to about 1:3 in certain embodiments, and a ratio of from about 1:0.4 to about 1:2 in certain embodiments. In certain embodiments, the acid can be l-glutamic acid, lactic acid, or citric acid.

In certain embodiments including a cationic surfactant, it can be beneficial to include a tertiary amine or quaternary amine. For example, in certain embodiments, suitable cationic surfactants can be a mono-alkyl or alkenyl amidoamine, a mono-alkyl or alkenyl ammonium salt, a di-alkyl ammonium salt, a PEG(n) alkylamine, or any combination thereof. The cationic alkyl or alkenyl chain length can be between 10 and 40 carbon atoms and can be branched or straight. In certain embodiments, the alkyl or alkenyl chain length can be 12 to 22 carbon atoms long. In certain embodiments, the alkyl or alkenyl chain length can be between 16 and 18 carbon atoms long.

In certain embodiments, a suitable cationic detersive surfactant can be selected from the group consisting of cetrimonium chloride, stearimonium chloride, behentrimonium chloride, behentrimonium methosulfate, behenamidopropyltrimonium methosulfate, stearamidopropyltrimonium chloride, arachidtrimonium chloride, and distearyldimonium chloride.

### C. Amphoteric Surfactants

In certain embodiments, a personal care composition can include a suitable amphoteric detersive surfactant. Generally any sulfate-free amphoteric surfactant known for use in hair care or other personal care compositions can be suitable. For example, amphoteric detersive surfactants suitable for inclusion in a personal care composition described herein can include those surfactants broadly described as derivatives of aliphatic secondary and tertiary amines in which the aliphatic radical can be straight or branched chain and wherein one of the aliphatic substituents contains from 8 to 18 carbon atoms and one aliphatic substituent contains an anionic group such as a carboxy, sulfonate, phosphate, or phosphonate group. In certain embodiments, suitable amphoteric detersive surfactants can include cocoamphoacetate, cocoamphodiacetate, lauroamphoacetate, lauroamphodiacetate, and mixtures thereof. Other suitable amphoteric surfactants include amidobetaines and amidosulfobetaines.

### D. Zwitterionic Surfactants

A personal care composition can, in certain embodiments, include a suitable zwitterionic detersive surfactant. For example, in certain embodiments, a personal care composition can include surfactants broadly described as derivatives of aliphatic quaternary ammonium, phosphonium, and sulfonium compounds, in which the aliphatic radicals can be straight or branched chain, and wherein one of the aliphatic substituents contains from 8 to 18 carbon atoms and one aliphatic substituent contains an anionic group such as carboxy, sulfonate, phosphate or phosphonate group. In certain embodiments, betaine zwitterionic surfactants, including high alkyl betaines, can be beneficial. In certain embodiments, a zwitterionic surfactant can alternatively, or additionally, be a sultaine surfactant. For example, hydroxysultaine surfactants such as cocamidopropyl hydroxysultaine can also be suitable.

Examples of betaine zwitterionic surfactants can include coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

### E. Non-Ionic Surfactants

In certain embodiments, a personal care composition can include a nonionic detersive surfactant. Generally, suitable nonionic surfactants can include compounds produced by the condensation of alkylene oxide groups (hydrophilic in nature) with an organic hydrophobic compound, which may be aliphatic or alkyl aromatic in nature. Examples of suitable nonionic detersive surfactants can include:
1. The polyethylene oxide condensates of alkyl phenols. For example, the condensation products of alkyl phenols having an alkyl group containing from 6 to 20 carbon atoms in either a straight chain or branched chain configuration, with ethylene oxide, the said ethylene oxide being present in amounts equal to from about 10 to about 60 moles of ethylene oxide per mole of alkyl phenol.
2. Those derived from the condensation of ethylene oxide with the product resulting from the reaction of propylene oxide and ethylene diamine products.
3. The condensation product of aliphatic alcohols having from 8 to 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from 10 to 14 carbon atoms.
4. Long chain tertiary amine oxides corresponding to the following general formula IIIA:

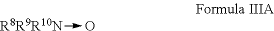

wherein R⁸ contains an alkyl, alkenyl or monohydroxy alkyl radical of from 8 to 18 carbon atoms, from 0 to about 10 ethylene oxide moieties, and from 0 to about 1 glyceryl moieties, and R⁹ and R¹⁰ contain from 1 to 3 carbon atoms and from 0 to about 1 hydroxy groups, e.g., methyl, ethyl, propyl, hydroxyethyl, or hydroxypropyl radicals. The arrow in the formula is a conventional representation of a semipolar bond.
5. Long chain tertiary phosphine oxides corresponding to the following general formula IIIB:

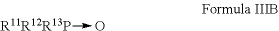

wherein R¹¹ contains an alkyl, alkenyl or monohydroxyalkyl radical ranging from 8 to 18 carbon atoms in chain length, from 0 to about 10 ethylene oxide moieties and from 0 to about 1 glyceryl moieties and R¹² and R¹³ are each alkyl or monohydroxyalkyl groups containing from 1 to 3 carbon atoms.
6. Long chain dialkyl sulfoxides containing one short chain alkyl or hydroxy alkyl radical of from 1 to 3 carbon atoms (usually methyl) and one long hydrophobic chain which include alkyl, alkenyl, hydroxy alkyl, or keto alkyl radicals containing from 8 to 20 carbon atoms, from 0 to about 10 ethylene oxide moieties and from 0 to about 1 glyceryl moiety.
7. Alkyl polysaccharide (“APS”) surfactants such as the alkyl polyglycosides. Such surfactants are described in U.S. Pat. No. 4,565,647 which is hereby incorporated by reference. APS surfactants can include a hydrophobic group with 6 to 30 carbon atoms and can include polysaccharide (e.g., polyglycoside) as the hydrophilic group. Optionally, there can be a polyalkylene-oxide group joining the hydrophobic and hydrophilic moieties. The alkyl group (i.e., the hydrophobic moiety) can be saturated or unsaturated, branched or unbranched, and unsubstituted or substituted (e.g., with hydroxy or cyclic rings).
8. Polyethylene glycol (PEG) glyceryl fatty esters, such as those of formula IIIC:

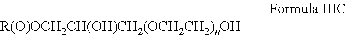

wherein n is from 5 to 200 or from 20 to 100, and R is an aliphatic hydrocarbyl having from 8 to 20 carbon atoms.
9. Glucoside surfactants including, for example, lauryl glucoside, coco glucoside, and decyl glucoside.
10. Certain surfactant-emulsifying compounds such as laureth-4.

In certain embodiments, specific examples of non-ionic detersive surfactants suitable for inclusion in a personal care composition can include cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

Additional examples of suitable detersive surfactants are described in McCutcheon's, Emulsifiers and Detergents, 1989 Annual, published by M. C. Publishing Co., U.S. Pat. Nos. 2,438,091, 2,528,378, 2,658,072, 3,929,678, 5,104,646, and 5,106,609, 6,649,155; U.S. Patent Application Publication No. 2008/0317698; and U.S. Patent Application Publication No. 2008/0206355, each of which are incorporated herein by reference.

The concentration of detersive surfactants in the personal care compositions can generally be selected to provide the desired cleaning and lather performance to the composition in combination with a dispersed gel network phase. In certain embodiments, a personal care composition can include, by weight, about 5% to about 50% of a detersive surfactant, in certain embodiments about 8% to about 30% of a detersive surfactant, in certain embodiments about 9% to about 25% of a detersive surfactant, and in certain embodiments about 10% to about 17% of a detersive surfactant.

### Liquid Carrier for the Personal Care Composition

The personal care composition also includes a liquid carrier separate from the liquid carrier of the dispersed gel network phase. Inclusion of an appropriate quantity of a liquid carrier can facilitate the formation of a personal care composition having an appropriate viscosity and rheology. A personal care composition can include, by weight of the composition, about 20% to about 95% of a liquid carrier in certain embodiments, and about 60% to about 85% of a liquid carrier in certain embodiments.

A liquid carrier can be water, or can be a miscible mixture of water and organic solvent. In certain embodiments however, a liquid carrier can be water with minimal or no significant concentrations of organic solvent, except as otherwise incidentally incorporated into the composition as minor ingredients of other essential or optional components. Suitable organic solvents can include water solutions of lower alkyl alcohols and polyhydric alcohols. Useful lower alkyl alcohols include monohydric alcohols having 1 to 6 carbons, such as ethanol and isopropanol. Exemplary polyhydric alcohols include propylene glycol, hexylene glycol, glycerin, and propane diol.

### Cationic Cellulose Polymers

In certain embodiments, a personal care composition can include a cationic cellulose polymer. A cationic polymer can be used to increase deposition or aid in the formation of a coacervate. In embodiments including a cationic polymer, the polymer can be included by weight of the personal care composition at about 0.05% to about 3%, about 0.075% to about 2.0%, or at about 0.1% to about 1.0%. In certain embodiments, cationic polymers can have cationic charge densities of about 0.9 meq/gm or more, about 1.2 meq/gm or more, and about 1.5 meq/gm or more. However, cationic charge density can also be about 7 meq/gm or less in certain embodiments and about 5 meq/gm or less in certain embodiments. The charge densities can be measured at the pH of intended use of the personal care composition. (e.g., at about pH 3 to about pH 9; or about pH 4 to about pH 8). In certain embodiments, the weight average molecular weight of the cationic polymer can be between about 10,000 g/mol and 10 million g/mol (e.g., 50,000 g/mol and about 5 million g/mol, and between about 100,000 g/mol and about 3 million g/mol).

In certain embodiments, suitable cationic polymers can contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Anionic counterions can be used in association with the cationic polymers so long as the polymers remain soluble in water, in the composition, and in a coacervate phase of the composition. Examples of suitable counterions include halide counterions (e.g., chloride, fluoride, bromide, iodide).

Other cationic polymers are also suitable including polysaccharide polymers, such as cationic cellulose derivatives and cationic starch derivatives. Suitable cationic polysaccharide polymers include those which conform to Formula IV:

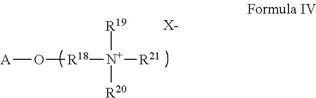

wherein A is an anhydroglucose residual group, such as a starch or cellulose anhydroglucose residual; R¹⁸ is an alkylene oxyalkylene, polyoxyalkylene, or hydroxyalkylene group, or combination thereof; R¹⁹, R²⁰, and R²¹ are independently alkyl, aryl, alkylaryl, arylalkyl, alkoxyalkyl, or alkoxyaryl groups, wherein each group contains up to 18 carbon atoms, and the total number of carbon atoms for each cationic moiety (i.e., the sum of carbon atoms in R¹⁹, R²⁰, and R²¹) is 20 or less; and X is an anionic counterion.

In certain embodiments, a cationic cellulose polymer can be selected from the salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable cationic cellulose polymers can include polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include copolymers of etherified cellulose, some examples of which are described in U.S. Pat. No. 3,958,581, which is incorporated herein by reference. Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

In embodiments including a cationic polymer, the cationic polymers can be soluble in the composition or can be soluble in a complex coacervate phase in the composition formed by interaction of the cationic polymer and a sulfate-free anionic, amphoteric or zwitterionic detersive surfactant. Complex coacervates of the cationic polymer can also be formed with other anionic charged materials in the personal care composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated herein by reference.

### Optional Components

As can be appreciated, personal care compositions described herein can include a variety of optional components to tailor the properties and characteristics of the composition. As can be appreciated, suitable optional components are well known and can generally include any components which are physically and chemically compatible with the essential components of the personal care compositions described herein. Optional components should not otherwise unduly impair product stability, aesthetics, or performance. Individual concentrations of optional components can generally range from about 0.001% to about 10%, by weight of a personal care composition.

In certain embodiments, examples of optional components which can be included in a personal care composition can include co-surfactants, deposition aids, cationic polymers, conditioning agents (including hydrocarbon oils, fatty esters, silicones), anti-dandruff agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, skin active agents, sunscreens, UV absorbers, and vitamins.

### Co-Surfactants

In certain embodiments, one or more co-surfactants can be included in a personal care composition to enhance various properties of a personal care composition. For example, a co-surfactant can improve the production of lather, facilitate easier rinsing, or further mitigate the harshness on detersive surfactants on keratinous tissue. In certain embodiments, a co-surfactant further can also aid in producing lather having more desirable textures and volume. In embodiments including co-surfactants, suitable co-surfactants can be selected from any of the sulfate-free amphoteric, zwitterionic, cationic, and nonionic surfactants previously disclosed as suitable detersive surfactants. When included, a co-surfactant can be included in a ratio with the detersive surfactant. For example, the ratio of the detersive surfactant to a co-surfactant can be about 1:20 to about 1:4 in certain embodiments, and a ratio of about 1:12 to about 1:7 in certain embodiments.

Alternatively, a co-surfactant can be included by weight percentage of the personal care composition. For example, a personal care composition can include a co-surfactant by weight of about 0.5% to about 10%, about 0.5% to about 5%, about 0.5% to about 3%, about 0.5% to about 2%, and about 0.5% to about 1.75%.

### Deposition Aid

In certain embodiments, a personal care composition can include a deposition aid to enhance deposition of the dispersed gel network phase. When included, a deposition aid can generally be selected from any material that enhances deposition of the gel network onto the hair and/or scalp. A deposition aid can be included at a concentration sufficient to effectively enhance the deposition of the gel network phase. In certain embodiments, a deposition aid can be included from about 0.05% to about 5% by weight, in certain embodiments, from about 0.075% to about 2.5% by weight, and in certain embodiments, from 0.1% to about 1.0%, by weight of the personal care composition. In certain embodiments, a deposition aid can be a cationic polymer.

### Nonionic Polymers

The personal care compositions can also optionally include nonionic polymers. For example, polyalkylene glycols having a molecular weight of more than about 1000 g/mol can be useful to include in a personal care composition. Polyalkylene glycols can have the general formula V:

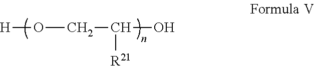

wherein R²¹ is selected from the group consisting of H, methyl, and mixtures thereof. Specific polyethylene glycol polymers which are suitable include PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### Conditioning Agents

Consumers sometimes prefer to purchase personal care compositions which provide both a cleaning benefit and a conditioning benefit. The inclusion of conditioning agents can allow for personal care compositions to be a “2 in 1” conditioning shampoo composition which both cleans and conditions hair. Generally, conditioning agents can include any material which provides a particular conditioning benefit to hair and/or skin. For example, in hair treatment compositions, suitable conditioning agents are those which deliver one or more benefits related to shine, softness, compatibility, antistatic properties, wet-handling, damage, manageability, body, and greasiness. In certain embodiments, suitable conditioning agents can include water insoluble, water dispersible, non-volatile, liquids that are emulsified into liquid particles or which are solubilized by the sulfate-free detersive surfactant. For example, a dimethicone or dimethiconol silicone agent can be included in certain embodiments.

In certain embodiments, suitable conditioning agents can generally include compounds classified as silicones or silicone derivatives (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the sulfate-free detersive surfactant. Generally any such conditioning agent can be suitable if it is physically and chemically compatible with the essential components of the personal care composition, and does not otherwise unduly impair product stability, aesthetics or performance.

The concentration of a conditioning agent in a personal care composition can be sufficient to provide the desired conditioning benefits. Such concentrations can vary with the conditioning agent, the conditioning performance desired, the average size of the conditioning agent particles, the type and concentration of other components, and other like factors. For example, inclusion of a cationic polymer to form a coacervate can improve the deposition of a conditioning agent. Optimization of conditioning agent concentration is generally known in the art.

### 1. Silicones

In embodiments where the conditioning agent is a silicone or silicone derivative, suitable silicone agents can include volatile silicone agents, non-volatile silicone agents, or a combination thereof. If volatile silicones are present, the volatile agents will typically be incidental to their use as a solvent or carrier for non-volatile silicone materials ingredients, such as silicone gums and resins. In certain embodiments, the silicone conditioning agent particles can include a silicone fluid conditioning agent and other ingredients, such as a silicone resin to improve silicone fluid deposition efficiency or enhance glossiness of the hair.

The concentration of a silicone conditioning agent can range from about 0.01% to about 10%, by weight of the composition, from about 0.1% to about 8%, from about 0.1% to about 5%, and from about 0.2% to about 3%. Non-limiting examples of suitable silicone conditioning agents, and optional suspending agents for the silicone, are described in U.S. Reissue Pat. No. 34,584, U.S. Pat. Nos. 5,104,646, and 5,106,609, each of which is hereby incorporated by reference herein. Suitable silicone conditioning agents can have a viscosity, as measured at about 25° C., from about 20 to about 2,000,000 centistokes (“csk”), from about 1,000 to about 1,800,000 csk, from about 50,000 to about 1,500,000 csk, and from about 100,000 to about 1,500,000 csk. The dispersed silicone conditioning agent particles can have a volume average particle diameter ranging from about 0.01 μm to about 50 μm. For small particle application to hair, the volume average particle diameters can range from about 0.01 μm to about 4 μm, from about 0.01 μm to about 2 μm, and from about 0.01 μm to about 0.5 μm. For larger particle application to hair, the volume average particle diameters can range from about 5 μm to about 125 μm, from about 10 μm to about 90 μm, from about 15 μm to about 70 μm, and from about 20 μm to about 50 μm.

Additional details about suitable silicone materials including silicone fluids, gums, and resins, as well as manufacture of silicones, are disclosed in Encyclopedia of Polymer Science and Engineering, vol. 15, 2d ed., pp 204-308, John Wiley & Sons, Inc. (1989), which is hereby incorporated by reference herein.

a. Silicone Oils

Suitable silicone fluids can include silicone oils which are flowable silicone materials having a viscosity, as measured at 25° C., or less than 1,000,000 csk, from about 5 csk to about 1,000,000 csk, and from about 100 csk to about 600,000 csk. Suitable silicone oils can include polyalkyl siloxanes, polyaryl siloxanes, polyalkylaryl siloxanes, polyether siloxane copolymers, and mixtures thereof. Other insoluble, non-volatile silicone fluids having hair conditioning properties can also be used.

For example, suitable silicone oils can include polyalkyl or polyaryl siloxanes which conform to Formula VI:

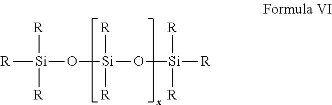

wherein each R is aliphatic, preferably alkyl or alkenyl, or aryl, R can be substituted or unsubstituted, and x is an integer from 1 to about 8,000. Suitable R groups can include alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Suitable alkyl and alkenyl substituents are C₁ to C₅ alkyls and alkenyls, C₁ to C₄ alkyls and alkenyls, and C₁ to C₂ alkyls and alkenyls. The aliphatic portions of other alkyl-, alkenyl-, or alkynyl-containing groups (such as alkoxy, alkaryl, and alkamino) can be straight or branched chains, and can be C₁ to C₅, C₁ to C₄, C₁ to C₃, or from C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length is preferably as described herein.

b. Amino and Cationic Silicones

Cationic silicone fluids suitable for use in the personal care compositions described herein can include silicone fluids described by general Formula VII:

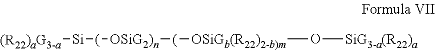

wherein G is hydrogen, phenyl, hydroxy, or C₁-C₈ alkyl, preferably methyl; a is 0 or an integer having a value from 1 to 3 or 0; b is 0 or 1; n is a number from 0 to 1,999 or 49 to 499; m is an integer from 1 to 2,000 or from 1 to 10; the sum of n and m is a number from 1 to 2,000, or from 50 to 500; R₂₂ is a monovalent radical conforming to the general formula CqH_{2q}L, wherein q is an integer having a value from 2 to 8 and L is selected from the following groups:

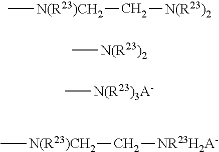

wherein R²³ is hydrogen, phenyl, benzyl, or a saturated hydrocarbon radical, preferably an alkyl radical from about C₁ to about C₂₀, and A is a halide ion.

In certain embodiments, an amino silicone can be a cationic silicone known as “trimethyl-silylamodimethicone”, which is shown below in Formula VIII:

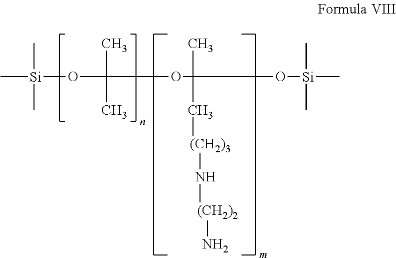

Other silicone cationic polymers which can be included include those described by Formula IX:

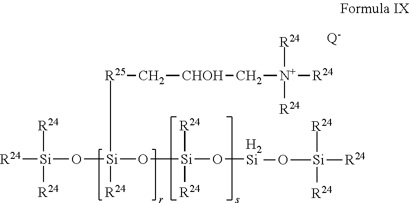

wherein R²⁴ is a monovalent hydrocarbon radical from C₁ to C₁₈ such as an alkyl or alkenyl radical, such as methyl; R²¹ is a hydrocarbon radical, such as a C₁ to C₁₈ alkylene radical or a C₁₀ to C₁₈ alkyleneoxy radical or a C₁ to C₈ alkyleneoxy radical; Q⁻ is a halide ion, such as chloride; r is an average statistical value from 2 to 20 or from 2 to 8; s is an average statistical value from 20 to 200 or from 20 to 50. An example of a suitable silicone cationic polymer of Formula IX is UCARE SILICONE ALE 56™, available from Union Carbide.
c. Silicone Gums

Other silicone fluids suitable for use in a personal care composition include insoluble silicone gums. Such gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, each of which are incorporated herein by reference. Specific examples of suitable silicone gums include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

d. High Refractive Index Silicones

Other non-volatile, insoluble silicone fluid conditioning agents that are suitable for use in a personal care composition include “high refractive index silicones,” having a refractive index of about 1.46 or more, about 1.48 or more, about 1.52 or more, and about 1.55 or more. The refractive index of the polysiloxane fluid can also generally be less than about 1.70, or less than about 1.60. In this context, polysiloxane “fluid” includes oils as well as gums.

The high refractive index polysiloxane fluid includes those represented by general Formula VI above, as well as cyclic polysiloxanes such as those represented by Formula X below:

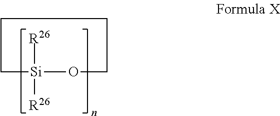

wherein R²⁶ is aliphatic, preferably alkyl or alkenyl, or aryl, R²⁶ can be substituted or unsubstituted, and n is a number from about 3 to about 7 or from about 3 to about 5.

A high refractive index polysiloxane fluid can contain an amount of aryl-containing R²⁶ substituents sufficient to increase the refractive index to the desired level. In certain embodiments, R²⁶ and n can be selected so that the material is non-volatile. Suitable aryl-containing substituents can include those which contain alicyclic and heterocyclic five and six member aryl rings and those which contain fused five or six member rings. The aryl rings themselves can be substituted or unsubstituted.

Generally, the high refractive index polysiloxane fluids can have a degree of aryl-containing substituents of about 15% or greater, about 20% or greater, about 25% or greater, about 35% or greater, or about 50% or greater. Typically, the degree of aryl substitution can be about 90% or less, and about 85% or less. In certain embodiments, the degree of aryl-containing substituents can vary from about 55% to about 80%.

In certain embodiments, high refractive index polysiloxane fluids can have a combination of phenyl or phenyl derivative substituents (more preferably phenyl), with alkyl substituents, preferably C₁-C₄ alkyl (more preferably methyl), hydroxy, or C₁-C₄ alkylamino (especially—R²⁷NHR²⁸NH2 wherein each R²⁷ and R²⁸ independently is a C₁-C₃ alkyl, alkenyl, and/or alkoxy).

When high refractive index silicones are included in a personal care composition, they are typically used in solution with a spreading agent, such as a silicone resin or a surfactant, to reduce the surface tension by a sufficient amount to enhance spreading and thereby enhance the glossiness (subsequent to drying) of hair treated with the compositions.

Suitable silicone fluids are also disclosed in U.S. Pat. Nos. 2,826,551, 3,964,500, 4,364,837, British Pat. No. 849,433, and Silicon Compounds, Petrarch Systems, Inc. (1984), all of which are incorporated herein by reference.

e. Silicone Resins

Silicone resins can be included in certain embodiments. As can be appreciated, such resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, a silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and are specifically defined for each occurrence.

In certain embodiments, suitable silicone resins can include MQ, MT, MTQ, MDT, and MDTQ resins. Methyl is a preferred silicone substituent. Especially preferred silicone resins are MQ resins, wherein the M:Q ratio is from about 0.5:1.0 to about 1.5:1.0 and the weight average molecular weight of the silicone resin is from about 1000 g/mol to about 10,000 g/mol.

The weight ratio of the non-volatile silicone fluid, having refractive index below 1.46, to the silicone resin component, when used, is from about 4:1 to about 400:1, from about 9:1 to about 200:1, from about 19:1 to about 100:1, particularly when the silicone fluid component is a poly-dimethylsiloxane fluid or a mixture of polydimethylsiloxane fluid and polydimethylsiloxane gum as described herein. Insofar as the silicone resin forms a part of the same phase in the compositions hereof as the silicone fluid, i.e., the conditioning active, the sum of the fluid and resin should be included in determining the level of silicone conditioning agent in the composition.

### 2. Organic Conditioning Oils

In certain embodiments, a conditioning component of a personal care composition can further include about 0.05% to about 3%, about 0.08% to about 1.5%, or 0.1% to about 1%, by weight of the composition, of at least one organic conditioning oil. The organic conditioning oil can be used either alone or in combination with other conditioning agents, such as a silicone.

a. Hydrocarbon Oils

Suitable organic conditioning oils can include hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils can be about C₁₂ to about C₁₉ in length in certain embodiments. Branched chain hydrocarbon oils, including hydrocarbon polymers, can contain more than 19 carbon atoms.

Specific examples of suitable hydrocarbon oils can include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which can include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers can include polybutene and polydecene. A hydrocarbon polymer can be polybutene, such as the copolymer of isobutylene and butene. A commercially available material of this type is L-14 polybutene from Amoco Chemical Corporation. The concentration of such hydrocarbon oils in the composition range from about 0.05% to about 20%, from about 0.08% to about 1.5%, and from about 0.1% to about 1%, by weight of the composition.

b. Polyolefins

Organic conditioning oils can also include liquid polyolefins such as liquid poly-α-olefins. In certain embodiments, a liquid polyolefin can be a hydrogenated liquid poly-α-olefins. Polyolefins for use herein are prepared by polymerization of C₄ to about C₁₄ or C₆ to about C₁₂ olefenic monomers.

Examples of suitable olefenic monomers can include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. In certain embodiments, it can be advantageous for the hydrogenated α-olefin monomers to be 1-hexene to 1-hexadecenes, 1-octene to 1-tetradecene, and mixtures thereof.

c. Fatty Esters

Other suitable organic conditioning oils can include fatty esters having at least 10 carbon atoms. Such fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof can include or have covalently bonded thereto other compatible functionalities, such as amides and alkoxy moieties (e.g., ethoxy or ether linkages, etc.).

Specific examples of suitable fatty esters can include: isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in a personal care composition can include mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for inclusion are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific examples of suitable di- and tri-alkyl and alkenyl esters of carboxylic acids can include isocetyl stearyol stearate, diisopropyl adipate, and tristearyl citrate.

Other fatty esters suitable are those known as polyhydric alcohol esters. Such polyhydric alcohol esters can include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, ethylene glycol distearate, polyoxyethylene polyol fatty acid ester, sorbitan fatty acid esters, and polyoxyethylene sorbitan fatty acid esters.

Still other fatty esters suitable for a personal care composition include glycerides. For example, mono-, di-, and tri-glycerides, preferably di- and tri-glycerides are suitable. In certain embodiments, the glycerides can be mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils can also be used including triolein and tristearin glyceryl dilaurate.

Other fatty esters suitable for use in the compositions of the present invention are water insoluble synthetic fatty esters described by Formula XI:

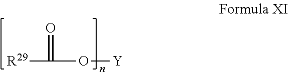

wherein R²⁹ is a C₇ to C₉ alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general Formula XII:

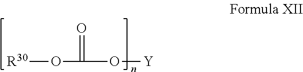

wherein R³¹ is a C₈ to C₁₀ alkyl, alkenyl, hydroxyalkyl or hydroxyalkenyl group; preferably a saturated alkyl group, more preferably a saturated, linear, alkyl group; n and Y are as defined above in Formula (XI).

In certain embodiments, synthetic fatty esters suitable for a personal care composition can include: P-43 (C₅-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

### 3. Other Conditioning Agents

As can be appreciated, a variety of additional conditioning agents are suitable as known in the art. For example, the conditioning agents described by the Procter & Gamble Company in U.S. Pat. Nos. 5,674,478, 5,750,122 are suitable and both patents are incorporated herein by reference. Also suitable are the conditioning agents described in U.S. Pat. Nos. 4,529,586, 4,507,280, 4,663,158, 4,197,865, 4,217,914, 4,381,919, and 4,422,853, each of which is incorporated herein by reference.

### Anti-Dandruff Actives

In certain embodiments, a personal care composition can also contain an anti-dandruff agent. Suitable anti-dandruff agents can include pyridinethione salts, azoles, selenium sulfide, particulate sulfur, and mixtures thereof. In certain embodiments, pyridinethione salts can be preferred. Such anti-dandruff particulate should be physically and chemically compatible with the essential components of the composition, and should not otherwise unduly impair product stability, aesthetics or performance. In certain embodiments, a personal care composition can include a cationic polymer to enhance deposition of an anti-dandruff active.

a. Pyridinethione Salts

In certain embodiments, an anti-dandruff agent can be a pyridinethione particulate such as a 1-hydroxy-2-pyridinethione salt. The concentration of pyridinethione anti-dandruff particulates can range from about 0.1% to about 4%, about 0.1% to about 3%, and from about 0.3% to about 2% by weight of the composition. Suitable pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form, wherein the particles have an average size of up to about 20μ, up to about 5μ, and up to about 2.5μ. Salts formed from other cations, such as sodium, can also be suitable. Pyridinethione anti-dandruff agents are further described in U.S. Pat. Nos. 2,809,971; 3,236,733; 3,753,196; 3,761,418; 4,345,080; 4,323,683; 4,379,753; and 4,470,982, each of which are incorporated herein by reference. It is contemplated that when ZPT is used as the anti-dandruff particulate, that the growth or re-growth of hair may be stimulated or regulated, or both, or that hair loss may be reduced or inhibited, or that hair may appear thicker or fuller.

b. Other Anti-Microbial Actives

In addition to the anti-dandruff active selected from polyvalent metal salts of pyrithione, a personal care composition can further include one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. In certain embodiments, preferred anti-microbials can include itraconazole, ketoconazole, selenium sulphide and coal tar.

c. Azoles

In certain embodiments, a suitable anti-microbial agent can be an azole. Examples of azole anti-microbials can include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in a personal care composition, the azole anti-microbial active can be included in an amount from about 0.01% to about 5%, from about 0.1% to about 3%, and more from about 0.3% to about 2%, by weight of the composition. In certain embodiments, a ketoconazole azole can be preferred.

d. Selenium Sulfide

Selenium sulfide is a particulate anti-dandruff agent suitable for use as an anti-microbial compositions when included at concentrations of about 0.1% to about 4%, by weight of the composition, from about 0.3% to about 2.5% by weight, and from about 0.5% to about 1.5% by weight. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. Nos. 2,694,668; 3,152,046; 4,089,945; and 4,885,107, each of which are incorporated herein by reference.

e. Sulfur

Sulfur can also be used as a particulate anti-microbial/anti-dandruff agent in certain embodiments. Effective concentrations of the particulate sulfur are typically from about 1% to about 4%, by weight of the composition, and from about 2% to about 4% by weight of the composition.

f. Keratolytic Agents

Keratolytic agents such as salicylic acid can also be included in a personal care composition described herein.

g. Other

Additional anti-microbial actives can include extracts of melaleuca (tea tree) and charcoal. As can be appreciated, personal care compositions can also include combinations of anti-microbial actives. Suitable combinations can include octopirox and zinc pyrithione combinations, pine tar and sulfur combinations, salicylic acid and zinc pyrithione combinations, octopirox and climbasole combinations, and salicylic acid and octopirox combinations, and mixtures thereof.

### Humectant

A personal care composition can also include a humectant to lower the rate of water evaporation. Suitable humectants can include polyhydric alcohols, water soluble alkoxylated nonionic polymers, and mixtures thereof. The humectants, when included, can be used at levels by weight of the composition of from about 0.1% to about 20%, and from about 0.5% to about 5%.

Suitable polyhydric alcohols can include glycerin, sorbitol, propylene glycol, butylene glycol, hexylene glycol, ethoxylated glucose, 1, 2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Suitable water soluble alkoxylated nonionic polymers can include polyethylene glycols and polypropylene glycols having a molecular weight of up to about 1000 g/mol such as those with CTFA names PEG-200, PEG-400, PEG-600, PEG-1000, and mixtures thereof.

### Suspending Agent

In certain embodiments, a personal care composition can include a suspending agent at concentrations effective for suspending water-insoluble material in dispersed form in the compositions or for modifying the viscosity of the composition. Such concentrations range from about 0.1% to about 10%, and from about 0.3% to about 5.0%, by weight of the compositions.

Suitable suspending agents can include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other suitable suspending agents can include crystalline suspending agents which can be categorized as acyl derivatives, long chain amine oxides, and mixtures thereof. Examples of such suspending agents are described in U.S. Pat. No. 4,741,855, which is incorporated herein by reference. Suitable suspending agents include ethylene glycol esters of fatty acids having from 16 to 22 carbon atoms. In certain embodiments, the suspending agent can be an ethylene glycol stearates, both mono and distearate, but particularly the distearate containing less than about 7% of the mono stearate. Other suitable suspending agents include alkanol amides of fatty acids, preferably having from 16 to 22 carbon atoms, more preferably 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the preferred materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

### Viscosity Modifiers

Viscosity modifiers can be used to modify the rheology of a personal care composition. Suitable viscosity modifiers can include carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol. Sodium chloride and sodium xylene sulfonate can also be used as a viscosity modifier. Other suitable rheology modifiers can include cross-linked acrylates, cross-linked maleic anhydride co-methylvinylethers, hydrophobically modified associative polymers, and mixtures thereof.

### Other Optional Components

As can be appreciated, a personal care composition can include still further optional components. For example, amino acids can be included. Suitable amino acids can include water soluble vitamins such as vitamins B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

In certain embodiments, a personal care composition can optionally include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The compositions can also include antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

In certain embodiments, one or more stabilizers and preservatives can be included. For example, one or more of trihydroxystearin, ethylene glycol distearate, citric acid, sodium citrate dihydrate, a preservative such as kathon, sodium chloride, sodium benzoate, and ethylenediaminetetraacetic acid (“EDTA”) can be included to improve the lifespan of a personal care compositon.

Chelants can also be included to scavenge metal and reduce hair damage caused by exposure to UV radiation. Examples of suitable chelants can include histidine and N,N′ethylenediamine disuccinic acid (“EDDS”).

### Multi-Phase Compositions

In certain embodiments, a personal care composition can include two or more phases to make a multiphase personal care composition. In such embodiments, one phase can include traditional personal care components, such as structured surfactants, and a second phase of the multiphase personal care compositions can include a benefit phase. Details of components and methods of making a multi-phase personal care composition are disclosed in U.S. Pat. No. 8,653,014 which is incorporated herein by reference.

### Method of Making a Personal Care Composition

A personal care composition described herein can be formed similarly to known personal care compositions. For example, the process of making a personal care composition can include (a) combining a fatty alcohol, a gel network surfactant, and water at a temperature sufficient to allow partitioning of the secondary surfactant and the water into the fatty alcohol to form a pre-mix; (b) cooling the pre-mix below the chain melt temperature of the fatty alcohol to form a gel network; (c) adding the gel network to one or more detersive surfactants and a liquid carrier to form a personal care composition which includes a dispersed gel network phase having a melt transition temperature of at least about 38° C.

In certain embodiments, a dispersed gel network phase can be formed as a separate pre-mix, which, after being cooled, can be subsequently incorporated with the other components of a personal care composition. More specifically, the gel network phase can be prepared by heating the fatty alcohol, the gel network surfactant, and water to a level in the range of about 75° C. to about 90° C. and mixing. This mixture can be cooled to a level in the range of about 27° C. to about 35° C. by, for example, passing the mixture through a heat exchanger. As a result of this cooling step, at least about fifty percent of the mixture of the fatty alcohol and the gel network surfactant crystallize to form a crystalline gel network.

Alternative methods of preparing the gel network phase include sonication and/or milling of the fatty alcohol, the gel network surfactant, and water, while these components are heated, to reduce the particle size of the dispersed gel network phase. This results in an increase in surface area of the gel network phase, which allows the gel network surfactant and the water to swell the gel network phase. Another suitable variation in preparing the gel network includes heating and mixing the fatty alcohol and the gel network surfactant first, and then adding that mixture to the water.

### Method of Use

The personal care compositions described herein can be used in a conventional manner for cleansing and conditioning of hair or skin. Generally, a method of treating hair or skin can include applying the personal care composition to the hair or skin. For example, an effective amount of the personal care composition can be applied to the hair or skin, which has been wetted with water, and then the composition can be rinsed off. Effective amounts can generally range from about 1 g to about 50 g in certain embodiments, and from about 1 g to about 20 g in certain embodiments. Application to the hair typically includes working the composition through the hair such that most or all of the hair is contacted with the composition.

In certain embodiments, a method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

In certain embodiments, a personal care composition as described herein can be used to treat damaged hair. Damaged hair can include hair permed hair, oxidatively colored hair, and mechanically damaged hair.

The personal care compositions can be used as liquids, solids, semi-solids, flakes, gels, in a pressurized container with a propellant added, or used in a pump spray form. The viscosity of the product may be selected to accommodate the form desired.

### Test Methods

### Hair Breakage Reduction via Shampoo Usage

Hair that is wavy and frizzy is contained into a 4 gram 30.5 cm long ponytail. Groups of 4 tresses of these are used for each treatment group, n=4. Hair tresses are weighed in a CTCH room prior to product application. Hair tresses are washed at a salon sink, with a flexible salon shower head; water conditions of 37-40 C, 6 ppm, and a water hardness between 6.5 and 7.5 gpg. A cycle of hair breakage study is as follows: wet the hair for 30 seconds, apply 0.1 cc/g of hair to the tress 1 cm below the binding in a zig-zag pattern to the front of the tress. Lather for 30 seconds using hands in a milking pattern, using the thumbs to massage the product into the tress fibers. Rinse for 30 seconds using a hand behind the tress to maintain water interaction. Using index and middle finger, draw down the tress to remove excess water. Hair is coarse combed 5 times by hand. Hair tress is transferred to a combing wheel that contains four fine-tooth combs (Cleopatra 400), that combs at a velocity of 13 RPM. Four revolutions of the wheel is completed creating a total of 16 comb strokes. Hair tress is then transferred to a wheel containing 4 brush heads of plastic back boars hair bristles. This wheel then contains a ladybird Conair hair dryer set at high velocity and high heat. Hair is brushed and blown dry for a total of 16 brush strokes with 90 seconds of blow drying. This completes one cycle of hair breakage study. A total of four cycles of hair breakage study are completed and hair is equilibrated for 12 hours and weighed. Hair loss is comparison of final cycle versus baseline.

Non-conditioning shampoo is used as a comparison control.

Anti-oxidant Response Element Assay

This method provides an ex vivo procedure for measuring the ability of a test agent or composition to activate the ARE in a cell. The method use cryopreserved passaged stocks of AREc32 cells, which contain a luciferase gene that fluoresces when the ARE is activated. The light emitted during fluorescence is detected and correlated to an activation level of the ARE. The AREc32 cells are expanded over 4-5 days in culture flasks and passaged every 3-4 days (when cells are˜80% confluent). When cells are >70% confluent or ready to seed into 96 well plates, the cells are trypsinized, seeded and grown in 96-well plates. After growing for 1 day in 96-well plates, media is replaced with fresh treatment media (phenol red free, no FBS) and cells are treated with compounds and incubated overnight (24 hours). Post treatment, cells are rinsed with 1×PBS, lysed and receive the luciferase kit reagent and luminescence measured.

### Assay Protocol Cell Preparation and Treatment:

1. In a 96 well-plate, seed 1.5×104 cells/well* in 100 μl DMEM (Dulbecco's Modified Eagle Medium) maintenance/plating media and grown overnight in cell culture incubator.
2. Let cells sit for 15 minutes at room temperature post seeding prior to placing in incubator to allow the cells to settle.
3. Incubate the cells at 37° C. in a 5% CO2, 95% humidity incubator for 24 hrs.
4. Replace the medium with 99 μl DMEM treatment media and treat with test compounds 4 ul per well, vehicle 4 ul per well and positive control.
5. Add additional 98 μl of media after treatment to wells, final assay volume 200 μl: Adding half the media after dosing insures better distribution of the materials.
6. Incubate the cells at 37° C. in CO2, 95% humidity incubator for another 24 hrs.
7. Remove media via aspiration and wash the cells once with 100 μl 1×PBS buffer. Remove PBS and follow instructions for luciferase assay.

### Luciferase Assay:

1. Prepare 1× lysis reagent by adding 4 volumes of water to 1 volume of 5× lysis reagent.
2. Add 20 μl of lysis buffer per well. Gently shake plate to distribute lysis buffer in well. Place plate in −80° C. freezer for 15 minutes to facilitate cell lysis. Thaw plate completely. Can confirm lysis under microscope.
3. Prepare Luciferase Assay Reagent (LAR) by adding Luciferase Assay Buffer (10 ml for E152A and 100 ml for E152B) to the vial of lyophilized Luciferase Assay Substrate. Mix gently. Add 100 μl Luciferase Assay Reagent per well. Ensure there are no air bubbles.
4. Read plate immediately using Neo2 plate reader or equivalent.

### Non-Limiting Examples

The personal care compositions illustrated in the following Examples illustrate specific embodiments of the personal care compositions described herein, but are not intended to be limiting thereof. Other modifications can be undertaken by the skilled artisan without departing from the spirit and scope of this invention. These exemplified embodiments of the personal care composition provide suitable cleaning benefits to hair without the use of a harsh sulfate-based surfactant.

The personal care compositions illustrated in the following Examples are prepared according to the formulation and mixing methods described herein alone with any conventional methods of making such compositions, as required. All exemplified amounts are listed as weight percent of active material and exclude minor materials such as diluents, preservatives, color solutions, imagery ingredients, botanicals, and so forth, unless otherwise specified. All percentages are based on weight unless otherwise specified. Several example compositions from Table 1 were tested according to the Hair Breakage Reduction via Shampoo Usage method to determine whether they provided a hair strength improvement benefit. The results of the testing are summarized in Table 2.

TABLE 1
Example Formula

| | Example | Example | Example | Example | Example | Example | Example | Example |
|---|---|---|---|---|---|---|---|---|
| Example | A | B | C | D | E | F | G | H |
| Lauramidopropyl Betaine | 10.0% | 10.0% | 10.0% | 8.0% | 8.0% | 8.0% | 8.0% | 8.0% |
| (35% active) | | | | | | | | |
| Isethionate | 6.0% | 6.0% | 6.0% | | | | | |
| Sarcosinate (30% active) | 2.0% | 2.0% | 0.0% | 4.8% | 4.8% | 4.8% | 4.8% | 4.8% |
| Dimethiconol (22% active) | 0.3% | 0.3% | — | 1.0% | 1.0% | 1.0% | 1.0% | 1.0% |
| Cationic Cellulose | 0.25% | 0.25% | 0.8% | — | — | — | — | — |
| RheoCareTTA (30% active) | — | — | 0.2% | 0.3% | 0.3% | 0.2% | 0.2% | 0.2% |
| EDTA (87% active) | 0.1% | 0.1% | 0.2% | 0.2% | 0.1% | 0.2% | 0.2% | 0.2% |
| Sodium Benzoate | 0.8% | 0.8% | 0.8% | 0.8% | 0.8% | 0.8% | 0.8% | 0.8% |
| Kathon (as is basis, 1.5%)) | — | — | 0.0033% | — | — | — | 0.02% | 0.02% |
| Sodium Salicylate | 0.5% | 0.5% | 0.5% | 0.5% | 0.5% | 0.5% | — | — |
| Fragrance | 1.0% | 1.0% | 1.0% | 1.0% | 1.0% | 1.0% | 1.0% | 1.0% |
| Aloe vera(ppm) | 0.03% | — | 0.03% | 0.03% | — | 0.03% | — | 0.03% |
| Sodium Citrate | 2.5% | 2.5% | 2.5% | 2.5% | 2.5% | 2.5% | 2.50% | 2.50% |

TABLE 2

| | Hair Breakage Reduction via | | |
|---|---|---|---|
| | Shampoo Usage 4 Cycles | | |
| | | Sig @ | |
| Example tested | Mean* | 95%** | p-Value |
| Example A | 1.11765 | A | 0.0135 |
| Example B | 1.57600 | B | |
| Example D | 1.32197 | A | 0.001 |
| Example E | 1.76115 | B | |
| Example F | 1.57368 | A | 0.0222 |
| Example G | 1.90367 | B | |
| Non-Conditioning Shampoo | 1.88810 | B | |

As can be seen in Table 2, the inventive compositions provide a hair strength as demonstrated by a reduction in hair breakage.

This example demonstrates the anti-oxidant benefit of aloe. Cell health for hair cells and skin cells depending in part on a balance of oxidants and anti-oxidants. When this balance is disturbed, undesirable damage can occur if the level of oxidants in the cell outweigh the anti-oxidant capacity of the cell. This damage affects the function of cellular proteins, membrane lipids, and DNA, and if unchecked, will result in impaired cell health.

Nuclear Factor Erythroid 2-related factor 2 (Nrf-2) is a transcription factor constitutively expressed in all tissues and is the principal transcription factor necessary for activating the anti-oxidant response element (ARE) of the cell. The greater the induction of the ARE, the greater the cell protection from oxidative damage. Examples A, B, D and E were tested according to the ARE assay. The results of the testing are summarized Table 3 below.

TABLE 3
Potent Aloe Shampoo Antioxidant Response Element (ARE)

| HE FB | | | |
|---|---|---|---|
| Shampoo - | | ARE Fold | Significant at |
| Aloe | HE Sulfate Free Potent Aloe | Activation | 95% CL vs |
| Level | Shampoo Formula | % | 0 ppm aloe* |
| 300 ppm | Example A | 2.43 | Yes |
| 0 ppm | Example B | 1.71 | N/A |
| 300 ppm | Example D | 2.53 | Yes |
| 0 ppm | Example E | 1.68 | N/A |

It will be appreciated that other modifications of the present disclosure are within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

What is claimed is:

1. A method of making a multi-phase personal care composition, comprising:
a) combining a fatty alcohol, a sulfate-free gel network surfactant and water such that the gel network surfactant and the water partition into the fatty alcohol to form a pre-mix;
b) cooling the pre-mix below the chain melt temperature of the fatty alcohol to form a gel network;
c) adding the gel network to a liquid carrier comprising water;
d) adding a sulfate-free detersive surfactant to the liquid carrier;
e) adding about 0.01% to about 0.05% aloe vera to the liquid carrier, based on the weight of the personal care composition, wherein the aloe vera comprises about 100 mg/100 g to about 300 mg/100 g of a salt selected from sodium salt, potassium salt, calcium salt, zinc salt, magnesium salt, copper salt and mixtures thereof;
f) adding about 0.1 wt % to about 3 wt % of a cationic cellulose polymer, based on the weight of the personal care composition; and
g) mixing the liquid carrier, gel network, detersive surfactant, aloe vera and cationic cellulose polymer to form the multi-phase personal care composition.

2. The method of claim 1, wherein the water is heated to at least about 74° C., and then combined with the fatty alcohol and gel network surfactant.

3. The method of claim 1, wherein fatty alcohol and gel network surfactant are heated to at least about 74° C. and then combined with the water.

4. The method of claim 1, wherein the combination of fatty alcohol, gel network surfactant and water is heated to a temperature of about 75° C. to about 90° C.

5. The method of claim 1, further comprising sonicating or milling the pre-mix prior to cooling.

6. The method of claim 1, wherein the pre-mix is cooled to a temperature of about 27° C. to about 32° C. to form the gel network.

7. The method of claim 5, wherein the gel network has a melt transition temperature of at least about 38° C.

8. The method of claim 1, wherein the multi-phase personal care composition provides a reduction in hair breakage according to the Hair Breakage Reduction via Shampoo Usage method.

9. The method of claim 1, wherein the cationic cellulose polymer conforms to the following formula: 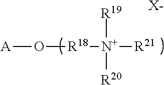
wherein A is an anhydroglucose residual group selected from starch, cellulose and mixtures thereof; R¹⁸ is an alkylene oxyalkylene, polyoxyalkylene, hydroxyalkylene or combination thereof; R¹⁹, R²⁰, and R²¹ are independently alkyl, aryl, alkylaryl, arylalkyl, alkoxyalkyl, or alkoxyaryl groups, wherein each group contains up to 18 carbon atoms; X− is an anionic counterion, and wherein the total number of carbon atoms in R¹⁹, R²⁰, and R²¹ is 20 or less.

10. The method of claim 9, wherein the cationic cellulose polymer has a charge density of about 0.4 to about 2.6.

11. The method of claim 9, wherein the cationic cellulose polymer has a weight average molecular weight of from about 1 million g/mol to about 2.2 million g/mol.

12. The method of claim 1, wherein the detersive surfactant and the gel network surfactant are each selected from the group consisting of an isethionate, a sarcosinate, a sulfonate, a sulfosuccinate, a sulfoacetate, a glycinate, a glutamate, an ester, a carboxylate, an amphoacetate, a taurate, an acyl amino acid, a betaine, a sultaine, or a mixture thereof.

13. The method of claim 1, wherein the detersive surfactant is added at about 5% or more, by weight of the personal care composition.

14. The method of claim 1, further comprising adding a stabilizing agent selected from trihydroxystearin, ethylene glycol distearate, citric, citrate, a preservative, sodium chloride, sodium benzoate, ethylenediaminetetraacetic acid (“EDTA”) or a salt thereof.

15. The method of claim 1, further comprising adding a silicone conditioning agent selected from dimethicone, dimethiconol, and an amino silicone.

16. The method of claim 1, further comprising adding a cationic surfactant that conforms to the following formula: 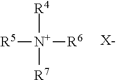
wherein at least one of R⁴, R⁵, R⁶ and R⁷ is selected from an aliphatic group of 8 to 30 carbon atoms or an aromatic, alkoxy, polyoxyalkylene, alkylamido, hydroxyalkyl, aryl or alkylaryl group having up to 22 carbon atoms, and wherein the remainder of R⁴, R⁵, R⁶ and R⁷ are independently selected from an aliphatic group having 1 to 22 carbon atoms or an aromatic, alkoxy, polyoxyalkylene, alkylamido, hydroxyalkyl, aryl or alkylaryl group having up to 22 carbon atoms; and X⁻ is a salt-forming anion selected from halogens, acetates, citrates, lactates, glycolates, phosphates, nitrates, sulfonates and alkyl sulfonate radicals.

17. The method of claim 1, wherein the multi-phase personal care composition is a shampoo.
